(19) United States
(12) Patent Application Publication
(10) Pub. No.: US 2023/0250481 A1
(43) Pub. Date: Aug. 10, 2023

(54) METHOD AND SYSTEM OF DIAGNOSING AND TREATING NEURODEGENERATIVE DISEASE AND SEIZURES

(71) Applicant: MOREHOUSE SCHOOL OF MEDICINE, Atlanta, GA (US)

(72) Inventors: Robert MELLER, Covington, GA (US); Roger P. SIMON, Atlanta, GA (US)

(21) Appl. No.: 18/160,373

(22) Filed: Jan. 27, 2023

Related U.S. Application Data
(62) Division of application No. 17/305,480, filed on Jul. 8, 2021.

(51) Int. Cl. C12Q 1/6883; C12Q 1/6806; C12Q 1/6809; A61K 45/06; C12Q 1/6869

(52) U.S. Cl. CPC C12Q 1/6883; C12Q 1/6806; C12Q 1/6809; A61K 45/06; C12Q 1/6869; C12Q 2600/112; C12Q 2600/166; C12Q 2600/178

(57) ABSTRACT

A method of distinguishing a subject with pre-clinical Alzheimer's disease from those with similar symptoms but other forms of dementia such as mild cognitive impairment. The blood RNA whole transcriptome profile of a subject with suspected pre-clinical Alzheimer's disease is obtained and analyzed against a reference blood RNA whole transcriptome profile from a subject with another form of dementia such as frontal temporal dementia, CADASIL or mild cognitive impairment (MCI). The blood RNA whole transcriptome profile includes the presence and quantitation of ncRNA. Methods to enhance treatment of epileptic seizures are also discussed.

This application is a Divisional application of U.S. application Ser. No. 17/305,480, filed Jul. 8, 2021. The entirety of which is incorporated herein by reference.

This application was made with government support under grants awarded by the NIH. The government has certain rights in the application.

### FIELD

The present disclosure relates to the treatment of neurodegenerative disease, in particular pre-clinical Alzheimer's disease and mild cognitive impairment, as well as treatment and prevention of seizures.

### BACKGROUND

Alzheimer's disease (“AD”) is a progressive neuro-degenerative condition that affects over 47 million people worldwide. AD is characterized by a loss in cognitive and memory function, and the formation of amyloid plaques and tau tangles in the brain. The pathological identification of amyloid plaques in the post mortem brain is usually accepted as the gold standard for AD diagnosis. One key clinical challenge of AD is to identify patients with Alzheimer's dementia vs. other forms of dementia. It is estimated that eight times as many people have preclinical AD versus AD per se, hence disease modifying agents are needed. This suggests that therapies targeting the mechanisms of AD may need to be administered earlier than the onset of cognitive impairment (i.e. before mild cognitive impairment (“MCI”)).

There has been a shift from use of a clinical diagnosis for AD to use of a biomarker diagnosis of AD. Current approaches are based on a bioassay of a cerebrospinal fluid sample obtained by lumbar puncture. As such a more accessible biofluid to obtain an accurate biomarker is needed.

Furthermore, there is no reliable test to differentiate epileptic seizure from various other conditions presenting as transient loss of consciousness. This can result in in appropriate modes of care being administered. Thus, there is a need for an accurate biomarker that can distinguish between patients who have an epileptic seizure (40%) from those whose seizure spells ae from syncope (25%), psychogenic non-epileptic seizures (PNES), or other non-epileptic spells (10%).

Blood is one of the most commonly assayed biofluids and satisfies NIH guidelines for biomarkers using accessible tissues. The present application is premised on the use of peripheral blood as the biofluid of choice.

### SUMMARY

An aspect of the application is a method of pre-clinical detection for incipient neurodegenerative disease, comprising the steps of: extracting a whole blood sample from a subject; preparing an RNA library from the whole blood sample; sequencing the RNA library; determining differential expression of a plurality of RNA sequences comprised within the RNA library, wherein the plurality of RNA sequences comprises both protein coding and non-coding RNA (ncRNA); creating a blood RNA transcriptome profile based on the differential expression of the RNA sequences; comparing the blood RNA transcriptome profile to a reference blood RNA transcriptome profile derived from a subject with neurodegenerative disease; detecting incipient neurodegenerative disease based on the correspondence between the blood RNA transcriptome profile and the reference profile derived from a subject with neurodegenerative disease. In one embodiment, the neurodegenerative disease is Alzheimer's disease. In a particular embodiment, the neurodegenerative disease is pre-clinical AD. In certain embodiments, the method further comprises the step of detecting pro-dromal Alzheimer's disease. In specific embodiments, the RNA library further comprises miRNA and mRNA. In other embodiments, the method further comprises comparing the blood RNA transcriptome profile to a reference blood RNA transcriptome profile from a subject with a dementia selected from one or more of the group consisting of frontal temporal dementia, CADASIL and mild cognitive impairment (MCI). In certain embodiments, the subject is selected based on one or more characteristics selected from the group consisting of geographical location, race, sex, age, weight, height (BMI), blood pressure, heartrate, body temperature, medications, routine admission blood studies and drug screens. In other embodiments, the neurodegenerative disease is one or more selected from the group consisting of Huntington's disease, Parkinson's disease, trinucleotide repeat disorders (DRPLA, SBMA, SCA1, SCA2, SCA3, SCA6, SCA7, SCA17, FRAXA, FXTAS, FRAXE, FRDA, DM1, SCA8, SCA12), amyotrophic lateral sclerosis and Batten disease.

Another aspect of the application is a method of enhancing treatment of preclinical Alzheimer's disease, comprising the steps of: extracting a whole blood sample from a subject; preparing an RNA library from the whole blood sample; sequencing the RNA library; determining differential expression of a plurality of RNA sequences comprised within the RNA library, wherein the plurality of RNA sequences comprises non-coding RNA (ncRNA); creating a blood RNA transcriptome profile based on the differential expression of the RNA sequences; comparing the blood RNA transcriptome profile to a reference blood RNA transcriptome profile derived from a subject with preclinical Alzheimer's disease; detecting preclinical Alzheimer's disease based on the correspondence between the blood RNA transcriptome profile and the reference profile derived from a subject with preclinical Alzheimer's disease; treating the subject with a therapy for Alzheimer's disease. In certain embodiments, the therapy for Alzheimer's disease comprises: administering cholinesterase inhibitors. In further embodiments, the cholinesterase inhibitors are selected from the group consisting of one or more of donepezil, rivastigimine and galantamine. In other embodiments, the therapy for Alzheimer's disease include antibody therapies, such as treatment with one or more of aducanumab, bapineuzumab, gantenerumab, crenezumab, BAN2401, GSK 933776, AAB-003, SAR228810, BIIB037/BART and solaneuzumab.

Another aspect of the application is a method of enhancing treatment of preclinical Parkinson's disease, comprising the steps of: extracting a whole blood sample from a subject; preparing an RNA library from the whole blood sample; sequencing the RNA library; determining differential expression of a plurality of RNA sequences comprised within the RNA library, wherein the plurality of RNA sequences comprises non-coding RNA (ncRNA); creating a blood RNA transcriptome profile based on the differential expression of the RNA sequences; comparing the blood RNA transcriptome profile to a reference blood RNA transcriptome profile derived from a subject with preclinical Parkinson's disease; detecting preclinical Parkinson's disease based on the correspondence between the blood RNA transcriptome profile and the reference profile derived from a subject with preclinical Parkinson's disease; treating the subject with a therapy for Parkinson's disease.

Another aspect of the application is a method of enhancing treatment of epileptic seizures, comprising the steps of: extracting a whole blood sample from a subject; preparing an RNA library from the whole blood sample; sequencing the RNA library; determining differential expression of a plurality of RNA sequences comprised within the RNA library, wherein the plurality of RNA sequences comprises non-coding RNA (ncRNA); creating a blood RNA transcriptome profile based on the differential expression of the RNA sequences; comparing the blood RNA transcriptome profile to a reference blood RNA transcriptome profile derived from a subject with an epileptic seizure; detecting epileptic seizure based on the correspondence between the blood RNA transcriptome profile and the reference profile derived from a subject with epileptic seizure; treating the subject with a therapy for epileptic seizure.

### BRIEF DESCRIPTION OF THE DRAWINGS

FIG. 1 shows analysis of RNA expression profiles distinguishes AD patients from healthy controls. Panel A. csf biomarker status of participants. AD and Controls subject to ROC analysis has an accuracy of 0.83. AUC for AD vs. other dementias was 0.53, and vs. MCI was 0.57 (not shown)(inset). Panel B. Hierarchical cluster analysis of differentially expressed genes shows a clear separation of healthy control (blue) and AD patient profiles (yellow). Panel C. Principal component analysis (PCA) shows clear separation of variability between samples.

FIG. 2 shows analysis of RNA expression profiles distinguishes AD patients from MCI patients. Panel A. Using the exon expression values identified above to distinguish AD from healthy controls, the PCA analysis was reperformed with the MCI data as well. Note how the MCI data fall in between the controls and AD patient groups. Panel B. A separate analysis of just MCI and AD patient groups was performed. Hierarchical cluster analysis of differentially expressed genes shows a clear separation of MCI and AD patient profiles. Panel C. Principal component analysis (PCA) shows clear separation of variability between patient groups.

FIG. 3 shows analysis of RNA expression profiles distinguishes AD patients from patients with other forms of dementia. In a separate analysis of AD and other forms of dementia (mixed), hierarchical cluster analysis of differentially expressed genes shows a clear separation of AD and other dementia patient profiles.

FIG. 4 shows use of post-seizure blood RNA profiles to develop an algorithm to retrospectively diagnose a seizure event. Blood samples from patients undergoing EEG monitoring are analyzed for RNA expression patterns at various times following seizure. These data are modeled to identify RNAs to predict the occurrence of a seizure, retrospectively. Panel A. Analysis of RNA expression profiles to African American pan genome, by race or ethnicity (AA—African American, CC Caucasian, HA Hispanic). Unique alignments make up 0.2% of mapped reads. Panel B. Comparison of alignment metrics of standard and de-nova transcriptome annotation guide generated using Blood RNA-seq data. More RNAs are called exons with the custom guide, and quantitated.

FIG. 5 shows the use of temporal blood RNA profiles to identify the nature of a seizure event. Blood samples from patients undergoing EEG monitoring are analyzed for RNA expression patterns at various times following seizure. These data are modeled to identify RNAs to predict the occurrence of a seizure, retrospectively.

While the present disclosure will now be described in detail, and it is done so in connection with the illustrative embodiments, it is not limited by the particular embodiments illustrated in the figures and the appended claims.

### DETAILED DESCRIPTION OF THE INVENTION

The invention and accompanying drawings will now be discussed in reference to the numerals provided therein to enable one skilled in the art to practice the present invention. The skilled artisan will understand, however, that the inventions described below can be practiced without employing these specific details, or that they can be used for purposes other than those described herein. Indeed, they can be modified and can be used in conjunction with products and techniques known to those of skill in the art considering the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

It will be appreciated that reference throughout this specification to aspects, features, advantages, or similar language does not imply that all the aspects and advantages may be realized with the present invention should be or are in any single embodiment of the invention. Rather, language referring to the aspects and advantages is understood to mean that a specific aspect, feature, advantage, or characteristic described in connection with an embodiment is included in at least one embodiment of the present invention. Thus, discussion of the aspects and advantages, and similar language, throughout this specification may, but do not necessarily, refer to the same embodiment.

The described aspects, features, advantages, and characteristics of the invention may be combined in any suitable manner in one or more further embodiments. Furthermore, one skilled in the relevant art will recognize that the invention may be practiced without one or more of the specific aspects or advantages of a particular embodiment. In other instances, additional aspects, features, and advantages may be recognized and claimed in certain embodiments that may not be present in all embodiments of the invention.

Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

Unless defined otherwise, a person skilled in the art understands all technical and scientific terms used herein to have the meaning commonly understood in the scientific and technical field. The following references are incorporated herein by reference: Singleton et al., Dictionary of Microbiology and Molecular Biology (2d ed. 1994); The Cambridge Dictionary of Science and Technology (Walker ed., 1988); The Glossary of Genetics, 5TH ED., R. Rieger et al. (eds.), Springer Verlag (1991); Hale & Marham, The Harper Collins Dictionary of Biology (1991); Chapter 3 of Laboratory Techniques in Biochemistry and Molecular Biology: Hybridization With Nucleic Acid Probes, Part I. Theory and Nucleic Acid Preparation, (P. Tijssen, ed.) Elsevier, N.Y. (1993); Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); Sambrook et al., Molecular Cloning: A Laboratory Manual, Cold Spring Harbor Press, N.Y., (1989); and Current Protocols in Molecular Biology, (Ausubel, F. M. et al., eds.) John Wiley & Sons, Inc., New York (1987-1999), including supplements such as supplement 46 (April 1999).

### Definitions

As used herein, the following terms have the meanings ascribed to them unless specified otherwise:

The term “tissue,” as used herein in the context of a source of nucleic acids, in particular RNA and cDNA, refers to an aggregation of cells that are morphologically or functionally related, or cell systems. Thus, in vitro cultured cells, as well as tissues, organs, and the like, are encompassed by the term tissue.

The term “library” as used herein, refers to a collection of polynucleotides derived from nucleic acid sequences of a particular tissue, in particular RNA or cDNA. The polynucleotides of a library may be, but are not necessarily, cloned into a vector or set in a microarray.

The terms “nucleic acid” “polynucleotide” and “oligonucleotide” may be used interchangeably herein and refer to a deoxyribonucleotide or ribonucleotide polymer in either single- or double-stranded form. A “subsequence” or “segment” refers to a sequence of nucleotides that comprise a part of a longer sequence of nucleotides.

A “gene,” for the purposes of the present disclosure, includes a DNA region encoding a gene product. The region can also include DNA regions that regulate the production of the gene product, whether or not such regulatory sequences are adjacent to coding and/or transcribed sequences. This term in science also encompasses RNAs which are expressed by a cell, but that are not translated into a protein, such as a non-coding RNA, micro RNA, piRNA, etc. Accordingly, a gene can include, without limitation, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA, or a novel RNA whose function is as yet to be determined) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

The term “transcriptome” refers to the set of all RNA molecules found in one cell or found in a population of cells. It is herein used to refer to all RNAs unless otherwise stated (e.g., the transcriptome is all RNA species, and their parts such as different isoforms (transcripts) and exons (small parts)). The transcriptome differs from the exome in that the transcriptome consists of only those RNA molecules contained in a specified cell population, and normally concerns the amount or concentration of each RNA molecule in addition to their molecular identities. The term can be applied to the whole set of transcripts in a given organism, or to a particular subset of transcripts found in a specific cell type. In contrast to the genome, the transcriptome can vary with external environmental conditions. Since the transcriptome comprises all RNA transcripts in the cell, the transcriptome reflects the active expression of different genes at any given time (although accounting for mRNA degradation phenomena, such as transcriptional attenuation, as an exception).

The term “neurodegenerative disease” refers to the progressive loss of structure or function of neurons, including death of neurons. Many neurodegenerative diseases—including amyotrophic lateral sclerosis, Parkinson's disease, Alzheimer's disease, and Huntington's disease occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously. There are many parallels between different neurodegenerative disorders including atypical protein assemblies as well as induced cell death. Neurodegeneration can be found in many different levels of neuronal circuitry ranging from molecular to systemic.

### Disorders

The term “Alzheimer's disease” refers to a chronic neurodegenerative disease characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyms.

The term “Parkinson's disease” refers to a long-term degenerative disorder of the central nervous system that mainly affects the motor system. The mechanism is by which the brain cells in Parkinson's are lost is not understood, but may consist of an abnormal accumulation of the protein alpha-synuclein bound to ubiquitin in the damaged cells. The alpha-synuclein-ubiquitin complex cannot be directed to the proteasome. This protein accumulation forms proteinaceous cytoplasmic inclusions called Lewy bodies. The latest research on pathogenesis of disease has shown that the death of dopaminergic neurons by alpha-synuclein is due to a defect in the machinery that transports proteins between two major cellular organelles—the endoplasmic reticulum (ER) and the Golgi apparatus. Certain proteins like Rabl may reverse this defect caused by alpha-synuclein in animal models.

The term “Amyotrophic lateral sclerosis” refers to a specific disease which causes the death of neurons controlling voluntary muscles. Some also use the term motor neuron disease for a group of conditions of which ALS is the most common. ALS is characterized by stiff muscles, muscle twitching, and gradually worsening weakness due to muscles decreasing in size. It may begin with weakness in the arms or legs, or with difficulty speaking or swallowing. About half of people develop at least mild difficulties with thinking and behavior and most people experience pain. Most eventually lose the ability to walk, use their hands, speak, swallow, and breathe.

The term “dementia” refers to a broad category of brain diseases that cause a long-term and often gradual decrease in the ability to think and remember that is great enough to affect a person's daily functioning. Other common symptoms include emotional problems, difficulties with language, and a decrease in motivation. A person's consciousness is usually not affected. A dementia diagnosis requires a change from a person's usual mental functioning and a greater decline than one would expect due to aging. The most common type of dementia is Alzheimer's disease, which makes up 50% to 70% of cases. Other common types include vascular dementia (25%), Lewy body dementia (15%), and frontotemporal dementia. Less common causes include normal pressure hydrocephalus, Parkinson's disease dementia, syphilis, and Creutzfeldt-Jakob disease among others. More than one type of dementia may exist in the same person. A small proportion of cases run in families. In the DSM-5, dementia was reclassified as a neurocognitive disorder, with various degrees of severity.

### Mild Cognitive Impairment

The term “mild cognitive impairment” refers to the first stages of dementia, the signs and symptoms of the disorder may be subtle. Often, the early signs of dementia only become apparent when looking back in time. The earliest stage of dementia is called mild cognitive impairment (MCI). 70% of those diagnosed with MCI progress to dementia at some point. In MCI, changes in the person's brain have been happening for a long time, but the symptoms of the disorder are just beginning to show. These problems, however, are not yet severe enough to affect the person's daily function. If they do, it is considered dementia. A person with MCI scores between 27 and 30 on the Mini-Mental State Examination (MMSE), which is a normal score. They may have some memory trouble and trouble finding words, but they solve everyday problems and handle their own life affairs well.

Diagnosis of MCI is often difficult, as cognitive testing may be normal. Often, more in-depth neuropsychological testing is necessary to make the diagnosis. The most commonly used criteria are called the Peterson criteria and include: memory or other cognitive (thought-processing) complaint by the person or a person who knows the patient well. The person must have a memory or other cognitive problem as compared to a person of the same age and level of education. The problem must not be severe enough to affect the person's daily function. The person must not have dementia.

Although MCI can present with a variety of symptoms, when memory loss is the predominant symptom it is termed “amnestic MCI” and is frequently seen as a prodromal stage of Alzheimer's disease. Studies suggest that these individuals tend to progress to probable Alzheimer's disease at a rate of approximately 10% to 15% per year.

### Preclinical Alzheimer's Disease

The term “preclinical Alzheimer's disease” refers to is a newly defined stage of the disease reflecting current evidence that changes in the brain may occur years before symptoms affecting memory, thinking or behavior can be detected by affected individuals or their physicians. Researchers currently use the term “preclinical Alzheimer's disease” to refer to the full spectrum from completely asymptomatic individuals with biomarker evidence of Alzheimer's to individuals manifesting subtle cognitive decline but who do not yet meet accepted clinical criteria for mild cognitive impairment (MCI).

The guidelines defining this stage were recommended by a workgroup, consisting of experts from the National Institute on Aging and the Alzheimer's Association. While these guidelines identify these preclinical changes as an Alzheimer's stage, they do not establish diagnostic criteria that doctors can use now. Rather, they propose additional research to establish which biomarkers may best confirm that dementia-related changes are underway in the brain, and how best to measure them. A biomarker is something that can be measured to accurately and reliably indicate the presence of disease. An example of a biomarker is fasting blood glucose (blood sugar) level, which indicates the presence of diabetes if it is 126 mg/dL or higher.

Emerging data in clinically normal older individuals suggest that amyloid plaque accumulation is associated with brain changes. Therefore, the long preclinical phase of Alzheimer's disease may provide a crucial window of opportunity to intervene with disease modifying treatment.

A recent report on the economic implications of the impending epidemic of Alzheimer's, as the “baby boomer” generation ages, suggests that 13.5 million individuals will get the disease by 2050. A hypothetical intervention that delayed the onset by 5 years would result in a 57% reduction in the number of Alzheimer's patients, and reduce the projected Medicare costs from $627 to $344 billion dollars.

Screening and treatment programs instituted for other diseases such as cholesterol screening for heart disease, colonoscopy for colon cancer, and mammography for breast cancer have already been associated with a decrease in mortality due to these conditions. The current lifetime risk of Alzheimer's disease for a 65-year-old is estimated to be 10.5%. Computer models suggest that a screening instrument for Alzheimer's, and an early treatment that slows progression by 50%, would reduce that risk to 5.7%.

Both laboratory work and recent disappointing clinical trial results raise the possibility that therapeutic interventions applied earlier in the disease would be more likely to modify its course. Studies with mice suggest that amyloid-modifying therapies may have limited impact once the degeneration of brain neurons has begun. Several recent clinical trials in the stages of mild to moderate dementia have failed to demonstrate clinical benefit, even with autopsy evidence of decreased amyloid plaque in the brain.

Several biomarker initiatives, including the Alzheimer's Disease Neuroimaging Initiative (ADNI), and the Australian Imaging, Biomarker & Lifestyle Flagship Study of Ageing (AIBL), as well as several major biomarker studies in the United States are ongoing. These studies have already provided preliminary evidence that biomarker abnormalities consistent with Alzheimer's disease are detectable prior to symptoms showing.

In other words, amyloid plaque build-up is present in the brains of the healthy individuals being studied. The number is dependent on their age and genetic background, but ranges from approximately 20-40%. Interestingly, the percentage of amyloid-positive normal individuals detected at a given age closely parallels the percentage of individuals diagnosed with Alzheimer's dementia a decade later.

People with preclinical Alzheimer's disease dementia may never experience any clinical symptoms during their lifetimes because of its long and variable preclinical period. By disease state, analysis showed that lifetime risks at each age increase by disease state in the following order: normal; neurodegeneration alone; amyloidosis alone; amyloidosis and neurodegeneration; mild cognitive impairment (MCI) with neurodegeneration; and MCI with amyloidosis and neurodegeneration. Researchers found that lifetime risks usually decrease with age for people in any given disease state: the lifetime risk for a woman aged 90 years with only amyloidosis is 8.4%, but is 29.3% for a woman aged 65 years with this same disease state. They noted the risk is greater for the younger patient because the 90-year-old has a shorter life expectancy than a 65 year old.

The researchers found that the presence of preclinical Alzheimer's disease does not always signal a high likelihood of Alzheimer's disease dementia. In addition, the results demonstrated that people aged younger than 85 years with MCI, amyloidosis and neurodegeneration carry a lifetime risk for Alzheimer's disease dementia of 50% or greater.

### Non-Coding RNA

At steady state, the vast majority of human cellular RNA consists of rRNA (˜90% of total RNA for most cells). Although there is less tRNA by mass, their small size results in their molar level being higher than rRNA. Other abundant RNAs, such as mRNA, snRNA, and snoRNAs are present in aggregate at levels that are about 1-2 orders of magnitude lower than rRNA and tRNA. Certain small RNAs, such as miRNA and piRNAs can be present at very high levels; however, this appears to be cell type dependent.

The term “non-coding RNA” (ncRNA) refers to an RNA molecule that is not translated into a protein. The number of non-coding RNAs within the human genome is unknown; however, recent transcriptomic and bioinformatics studies suggest that there are thousands of them. Many of the newly identified ncRNAs have not been validated for their function. It is also likely that many ncRNAs are non functional (sometimes referred to as junk RNA), and are the product of spurious transcription. Abundant and functionally important types of non-coding RNAs include transfer RNAs (tRNAs) and ribosomal RNAs (rRNAs), as well as small RNAs such as microRNAs, siRNAs, piRNAs, snoRNAs, snRNAs, exRNAs, scaRNAs and the long ncRNAs such as Xist and HOTAIR. The ncRNA may have some associated activity that may be deleterious. Most often the major concern is whether it will be translated into short random peptides.

The eukaryotic genome produces a vast amount of spurious transcripts. The existence of ncRNAs in significant amounts may contribute a burden to the cell. By general convention, most ncRNAs longer than 200 nucleotides, regardless of whether or not they have a known function, have been lumped together into a category called “long non-coding RNAs” (lncRNAs). There are an estimated 21,000 human lncRNAs, with an average length of about 1 kb. As a whole, lncRNAs are present at levels that are two orders of magnitude less than total mRNA.

Short ncRNAs include miRNA, siRNA, short enhancer RNAs (eRNAs), circular RNAs and piRNA. MicroRNAs (miRNA) generally bind to a specific target messenger RNA with a complementary sequence to induce cleavage, or degradation or block translation. Short interfering RNAs (siRNA) function in a similar way as miRNAs to mediate post-transcriptional gene silencing (PTGS) as a result of mRNA degradation. Piwi-interacting RNAs (piRNA) are so named due to their interaction with the piwi family of proteins. The primary function of these RNA molecules involves chromatin regulation and suppression of transposon activity in germline and somatic cells. PiRNAs that are antisense to expressed transposons target and cleave the transposon in complexes with PIWI-proteins. This cleavage generates additional piRNAs which target and cleave additional transposons. This cycle continues to produce an abundance of piRNAs and augment transposon silencing.

### Transcriptomics

Transcriptomic techniques include DNA microarrays and RNA-Seq. All transcriptomic methods require RNA to first be isolated from the experimental organism before transcripts can be recorded. Although biological systems are incredibly diverse, RNA extraction techniques are broadly similar and involve mechanical disruption of cells or tissues, disruption of RNase with chaotropic salts, disruption of macromolecules and nucleotide complexes, separation of RNA from undesired biomolecules including DNA, and concentration of the RNA via precipitation from solution or elution from a solid matrix. Isolated RNA may additionally be treated with DNase to digest any traces of DNA. Transcription can also be studied at the level of individual cells by single-cell transcriptomic.

RNA-Sequencing

The term “RNA-Seq” (RNA sequencing) refers to RNA-Seq (RNA sequencing), sometimes also referred to as whole transcriptome shotgun sequencing (WTSS). RNA-Seq uses high-throughput sequencing to illuminate the existence and relative quantities of RNA molecules at a given moment in a biological sample. RNA-Seq is used to study the continuously changing cellular transcriptome. In particular, RNA-Seq enables overview in different groups or treatments of alternative gene spliced transcripts, post-transcriptional modifications, gene fusion, mutations/SNPs and changes in gene expression over time, or differences in gene expression. In addition to mRNA transcripts, RNA-Seq can also look at different populations of RNA to include the whole RNS transcriptome (such as miRNA or tRNA). RNA-seq can be performed by single cell sequencing and also in situ sequencing of fixed tissue.

RNA-Seq works in concert with a range of high-throughput DNA sequencing technologies. However, prior to sequencing of the extracted RNA transcripts, several key processing steps are performed. Methods differ in the use of transcript enrichment, fragmentation, amplification, single or paired-end sequencing, and whether to preserve strand information. One of ordinary skill will understand that the particular type or form of RNA-Seq is not limiting on the invention discussed herein.

A variety of parameters is considered when designing and conducting RNA-Seq experiments:

Tissue specificity: Gene expression varies within and between tissues, and RNA-Seq measures this mix of cell types. This may make it difficult to isolate the biological mechanism of interest. Single cell sequencing can be used to study each cell individually, mitigating this issue.

Time dependence: Gene expression changes over time, and RNA-Seq only takes a snapshot. Time course experiments can be performed to observe changes in the transcriptome.

Coverage (also known as depth): RNA harbors the same mutations observed in DNA, and detection requires deeper coverage. With high enough coverage, RNA-Seq can be used to estimate the expression of each allele. This may provide insight into phenomena such as imprinting or cis-regulatory effects. The depth of sequencing required for specific applications can be extrapolated from a pilot experiment.

Data generation artifacts (also known as technical variance): The reagents (e.g., library preparation kit), personnel involved, and type of sequencer (e.g., Ion Torrent, Oxford Nanopore, Illumina, Pacific Biosciences) can result in technical artifacts that might be mis-interpreted as meaningful results. As with any scientific experiment, it is prudent to conduct RNA-Seq in a well controlled setting. If this is not possible or the study is a meta-analysis, another solution is to detect technical artifacts by inferring latent variables (typically principal component analysis or factor analysis) and subsequently correcting for these variables.

Data management: A single RNA-Seq experiment in humans is usually on the order of 1 Gb. This large volume of data can pose storage issues. One solution is compressing the data using multi-purpose computational schemas (e.g., gzip) or genomics-specific schemas. The latter can be based on reference sequences or de novo. Another solution is to perform microarray experiments, which may be sufficient for hypothesis-driven work or replication studies (as opposed to exploratory research).

In the case of blood, extracts may be typically 40-60% ribosomal RNA; in such cases, rRNA is not removed nor is the extract enriched for mRNA, which increases sample to sample variability; ncRNA is also not enriched and blood extracts are used as is, ncRNA may be high expression in blood.

In certain cases, it is necessary to enrich messenger RNA as total RNA extracts may be typically 98% ribosomal RNA. Enrichment for transcripts can be performed by poly-A affinity methods or by depletion of ribosomal RNA using sequence-specific probes. Degraded RNA may affect downstream results; for example, mRNA enrichment from degraded samples will result in the depletion of 5′ mRNA ends and an uneven signal across the length of a transcript. Snap-freezing of tissue prior to RNA isolation is typical, and care is taken to reduce exposure to RNase enzymes once isolation is complete.

The sensitivity of any given RNA-Seq analysis can be enhanced by enriching RNA classes of interest, while depleting known abundant RNAs. If so desired, the mRNA molecules can be removed by using oligonucleotides probes that bind their poly-A tails. Alternatively, abundant but uninformative ribosomal RNAs (rRNAs) are removed by ribo-depletion by hybridisation to probes designed to target specific rRNA sequences (e.g. mammal rRNA, plant rRNA). However, ribo-depletion can also introduce some bias via non-specific depletion of off-target transcripts, so is not preferred for the methods herein. Gel electrophoresis and extraction can be used to purify small RNAs, such as micro RNAs, by their size.

RNA transcripts are frequently fragmented prior to sequencing. Fragmentation may be achieved by chemical hydrolysis, nebulisation, sonication, or reverse transcription with chain-terminating nucleotides. Alternatively, fragmentation and cDNA tagging may be done simultaneously by using transposase enzymes. One of ordinary skill will understand that the particular method of preparing a transcriptome for sequencing is not limiting on the invention discussed herein.

During preparation for sequencing, cDNA copies of transcripts may be amplified by PCR to enrich for fragments that contain the expected 5′ and 3′ adapter sequences. Amplification is also used to allow sequencing of very low input amounts of RNA, down to as little as 50 pg in extreme applications. Spike-in controls of known RNAs can be used for quality control assessment to check library preparation and sequencing, in terms of QC-content, fragment length, as well as the bias due to fragment position within a transcript. Unique molecular identifiers (UMis) are short random sequences that are used to individually tag sequence fragments during library preparation so that every tagged fragment is unique. UMis provide an absolute scale for quantification, the opportunity to correct for subsequent amplification bias introduced during library construction, and accurately estimate the initial sample size. UMis are particularly well-suited to single-cell RNA-Seq transcriptomics, where the amount of input RNA is restricted and extended amplification of the sample is required.

Once the transcript molecules have been prepared they can be sequenced in just one direction (single-end) or both directions (paired-end). A single-end sequence is usually quicker to produce, cheaper than paired-end sequencing and sufficient for quantification of gene expression levels. Paired-end sequencing produces more robust alignments/assemblies, which is beneficial for gene annotation and transcript isoform discovery. Strand-specific RNA-Seq. methods preserve the strand information of a sequenced transcript. Without strand information, reads can be aligned to a gene locus but do not inform in which direction the gene is transcribed. Stranded-RNA-Seq is useful for deciphering transcription for genes that overlap in different directions and to make more robust gene predictions in non-model organisms. One of ordinary skill will understand that the particular strands used in sequencing are not limiting on the invention described herein.

Transcriptome Assembly

Transcriptomics methods are highly parallel and require significant computation to produce meaningful data for both microarray and RNA-Seq experiments. RNA-Seq analysis generates a large volume of raw sequence reads which have to be processed to yield useful information. Data analysis usually requires a combination of bioinformatics software tools that vary according to the experimental design and goals. The process can be broken down into four stages: quality control, alignment, quantification, and differential expression. Most popular RNA-Seq programs are run from a command-line interface, either in a Unix environment or within the R/Bioconductor statistical environment.

Sequence reads are not perfect, so the accuracy of each base in the sequence needs to be estimated for downstream analyses. Raw data is examined to ensure: quality scores for base calls are high, the GC content matches the expected distribution, short sequence motifs (k-mers) are not over-represented, and the read duplication rate is acceptably low. Several software options exist for sequence quality analysis, including FastQC and FaQCs. Abnormalities may be removed (trimming) or tagged for special treatment during later processes.

In order to link sequence read abundance to the expression of a particular RNA, transcript sequences are aligned to a reference genome or de novo aligned to one another if no reference is available. The key challenges for alignment software include sufficient speed to permit billions of short sequences to be aligned in a meaningful timeframe, flexibility to recognize and deal with intron splicing of eukaryotic mRNA, and correct assignment of reads that map to multiple locations. Software advances have greatly addressed these issues, and increases in sequencing read length reduce the chance of ambiguous read alignments. One of ordinary skill will understand the choice of high-throughput sequence aligners that are available and may be selected for analyses.

Alignment of primary transcript mRNA sequences derived from eukaryotes to a reference genome requires specialized handling of intron sequences, which are absent from mature mRNA. Short read aligners perform an additional round of alignments specifically designed to identify splice junctions, informed by canonical splice site sequences and known intron splice site information. Identification of intron splice junctions prevents reads from being misaligned across splice junctions or erroneously discarded, allowing more reads to be aligned to the reference genome and improving the accuracy of gene expression estimates. Since gene regulation may occur at the mRNA isoform level, splice-aware alignments also permit detection of isoform abundance changes that would otherwise be lost in a bulked analysis.

There are two general methods of inferring transcriptome sequences. One approach maps sequence reads onto a reference genome, either of the organism itself (whose transcriptome is being studied) or of a closely related species. Microarray data is recorded as high-resolution images, requiring feature detection and spectral analysis. Microarray raw image files are each about 750 MB in size, while the processed intensities are around 60 MB in size. Multiple short probes matching a single transcript can reveal details about the intron-exon structure, requiring statistical models to determine the authenticity of the resulting signal. RNA-Seq studies produce billions of short DNA sequences, which must be aligned to reference genomes composed of millions to billions of base pairs.

The other approach, de novo transcriptome assembly, uses software to infer transcripts directly from short sequence reads. De nova assembly of reads within a dataset requires the construction of highly complex sequence graphs. RNA-Seq operations are highly repetitious and benefit from parallelized computation but modern algorithms mean consumer computing hardware is sufficient for simple transcriptomics experiments that do not require de novo assembly of reads. One of ordinary skill will understand that the type of hardware is not limiting on the invention discussed herein.

De novo assembly can be used to align reads to one another to construct full-length transcript sequences without use of a reference genome. Challenges particular to de novo assembly include larger computational requirements compared to a reference-based transcriptome, additional validation of gene variants or fragments, and additional annotation of assembled transcripts. The metrics used to describe transcriptome assemblies are known to one of ordinary skill in the art. Annotation-based metrics may be used to assess assembly completeness (e.g. contig reciprocal best hit count). Once assembled de nova, the assembly can be used as a reference for subsequent sequence alignment methods and quantitative gene expression analysis. Challenges when using short reads for de novo assembly include 1) determining which reads should be joined together into contiguous sequences (contigs), 2) robustness to sequencing errors and other artifacts, and 3) computational efficiency. The primary algorithm used for de novo assembly transitioned from overlap graphs, which identify all pair wise overlaps between reads, to de Bruijn graphs, which break reads into sequences of length k and collapse all k-mers into a hash table. Overlap graphs were used with Sanger sequencing, but do not scale well to the millions of reads generated with RNA-Seq. Examples of assemblers that use de Bruijn graphs are Velvet, Trinity, Oases, and Bridger. Paired end and long read sequencing of the same sample can mitigate the deficits in short read sequencing by serving as a template or skeleton. Metrics to assess the quality of a de novo assembly include median contig length, number of contigs and N50.

Quantification of sequence alignments may be performed at the gene, exon, or transcript level. Typical outputs include a table of read counts for each feature supplied to the software; for example, for genes in a general feature format file. Gene and exon read counts may be calculated quite easily using HTSeq, for example. Quantitation at the transcript level is more complicated and requires probabilistic methods to estimate transcript isoform abundance from short read information; for example, using cufflinks software. Reads that align equally well to multiple locations must be identified and either removed, aligned to one of the possible locations, or aligned to the most probable location.

Some quantification methods can circumvent the need for an exact alignment of a read to a reference sequence altogether. The kallisto software method combines pseudoalignment and quantification into a single step that runs two orders of magnitude faster than contemporary methods such as those used by tophat/cufflinks software, with less computational burden.

Once quantitative counts of each transcript are available, differential gene expression is measured by normalizing, modelling, and statistically analyzing the data. Most tools will read a table of genes and read counts as their input, but some programs, such as cuffdiff, will accept binary alignment map format read alignments as input. The final outputs of these analyses are gene lists with associated pair-wise tests for differential expression between treatments and the probability estimates of those differences.

A genome-guided approach relies on the same methods used for DNA alignment, with the additional complexity of aligning reads that cover non-continuous portions of the reference genome. These non-continuous reads are the result of sequencing spliced transcripts. Typically, alignment algorithms have two steps: 1) align short portions of the read (i.e., seed the genome), and 2) use dynamic programming to find an optimal alignment, sometimes in combination with known annotations. Software tools that use genome-guided alignment include Bowtie, TopHat (which builds on BowTie results to align splice junctions), Subread, STAR, HISAT2, Sailfish, Kallisto, and GMAP. The quality of a genome guided assembly can be measured with both 1) de novo assembly metrics (e.g., N50) and 2) comparisons to known transcript, splice junction, genome, and protein sequences using precision, recall, or their combination (e.g., F1 score). In addition, in silico assessment could be performed using simulated reads.

For example, a human transcriptome could be accurately captured using RNA-Seq with 30 million 100 bp sequences per sample. This example would require approximately 1.8 gigabytes of disk space per sample when stored in a compressed fastq format. Processed count data for each gene would be much smaller, equivalent to processed microarray intensities. Sequence data may be stored in public repositories, such as the Sequence Read Archive (SRA). RNA-Seq datasets can be uploaded via the Gene Expression Omnibus, or similar software platforms. Treatment

Treatment

Distinguishing between preclinical Alzheimer's disease and mild cognitive impairment enables the selection and implementation of appropriate therapies at a very early stage of the disease, thereby delaying, and perhaps preventing, progression of the disease towards full-blown AD. In particular, treatment with cholinesterase inhibitors can begin once preclinical AD has been identified, e.g., donepezil, rivastigimine, galantamine. Treatments may also begin to address behavioral issues such as irritability, anxiety or depression. Antidepressants may include one or more drugs such as citalopram, fluoxetine, paroxetine, sertraline and trazodone. Anxiolytics may include one or more drugs such as lorazepam and oxazepam. Antipsychotics may include one or more drugs such as aripiprazole, clozapine, haloperidol, olanzapine, quetiapine, risperidone and ziprasidone. Other drugs for mood stabilization may include carbamazepine. Treatments for sleep changes may include one or more drugs such as tricyclic antidepressants (e.g. nortriptyline, trazodone), benzodiazepines (e.g., lorazepam, oxazepam and temazepam), zolpidem, zaleplon, chloral hydrate, risperidone, onlanzapine, quetiapine, and haloperidol. Other therapies may include one or more such as caprylic acid and coconut oil, coenzyme Q10, coral calcium, Ginkgo biloba, huperzine A, omega-4 fatty acids, phosphatidylserine, and tramiprosate.

One of ordinary skill will understand that treatments for other very early stages of neurodegenerative diseases, such as Parkinson's disease, may also be used when those diseases are identified by the RNA transcriptome profiles discussed herein. Treatments for Parkinson's disease may include one or more drugs such as levodopa, carbidopa, dopamine agonists, catechol O-methyltransferase (COMT) inhibitors, anticholinergics, amantadine and monoamine oxidase type B (MAO-B) inhibitors.

Epileptic Seizures

The present methods disclosed herein may also be used to distinguish epileptic seizures from other types of seizure, and thus enhance treatment effectiveness. Seizures, or spells, often frequently present as episodic transient loss of consciousness (TLOC). A major clinical challenge is to distinguish between patients who have an epileptic seizure (nearly 40%) from those whose spells are from syncope (25%), psychogenic non-epileptic seizures (PNES), or other non-epileptic spells (10%). The diagnosis of epileptic seizures is based on history with the semiology of events and clinical examination. However, extensive testing is often required with long-term clinical and electroencephalogram (EEG) monitoring to capture spells and characterize their electrographic pattern. This approach is time and resource consuming, frequently requires prolonged monitoring to successfully capture a spell of interest and in 35% of admissions, and results remain inconclusive. All these factors result in delays in definite diagnosis, leading to recurrent hospitalizations, and unnecessary treatments thereby incurring huge healthcare costs. Despite this comprehensive approach to evaluation of spells, misdiagnoses are not uncommon. To date, there is no reliable test to differentiate epileptic seizure from various other conditions presenting as transient loss of consciousness, unveiling a critical knowledge gap.

Application of the presently disclosed methods demonstrate that unique RNAs in whole blood samples persist 24 h following epileptic seizure termination. This enables discrimination between an epileptic seizure and non-epileptic seizures; this in turn can lead to more effective selection of treatments and therapies for both kinds of seizure.

Population studies have reported the incidence of epilepsy in both sexes is 44 cases per 100,000 person years. The incidence in females, at 41 cases per 100,000 person years, is less than that for males, at 49 cases per 100,000 person years. One epilepsy study also found that the prevalence of epilepsy was slightly higher in males than females (6.5 vs 6.0 per 1000 persons). As these higher rates in males may be attributable to the higher frequency of some major etiologies of seizures in men (e.g., cerebrovascular disease, head trauma, alcohol-related seizures), it may be that increasing rates of such conditions in women may result in less difference between the sexes. The risk for recurrent seizure is similar between males and females, as is the likelihood of ultimate remission of epilepsy. Although most epilepsy syndromes are equally or more commonly found in males than in females, childhood absence epilepsy and the syndrome of photosensitive epilepsy are more common in females. In addition, some genetic disorders with associated epilepsy (e.g., Rett syndrome and Aicardi syndrome) and eclamptic seizures in pregnancy can only occur in females. The methods disclosed herein can be applied with respect to the sex, whether male or female, of a patient, so as to enhance care with respect to sex (identified gender).

A study has shown that there is a significant association between epilepsy, race, and socioeconomic indicators in multivariate analysis. People identified as having genetic descent that is African had a prevalence rate for lifetime epilepsy that was 1.74 times higher than that of people identified as having genetic descent that is European after adjusting for age, education and income, and a corresponding prevalence rate for active epilepsy that was twice that for people identified as having genetic descent that is European. The methods disclosed herein can be applied with respect to the identified genetic descent of a patient whether African, Asian or European, so as to enhance care with respect to identified genetic descent.

Epilepsy can be treated by either medications, implanted devices, diet, surgery or a combination of these therapies. Most people are able to control the seizures caused by their epilepsy with medications called anti-epileptic drugs or AEDs. The type and severity of the seizure will determine what and how much medication is needed. The treatment for epileptic seizures. may comprise: administering one or more drugs selected from the group consisting of brivaracetam, ezogabine, pregabalin, cannabidiol oral solution, felbamate, primidone, carbamazepine, fenfluramine, rufinamide, carbamazepine-XR, gabapentin, stiripentol, cenobamate, lacosamide, tiagabine hydrochloride, lamotrigine, clobazam, levetiracetam, topiramate, clonazepam, levetiracetam XR, topiramate XR, diazepam nasal, lorazepam, valproic acid, diazepam rectal, oxcarbazepine, vigabatrin, divalproex sodium-ER, phenobarbital, eslicarbazepine acetate, phenytoin and ethosuximide. One of ordinary skill in the art will understand the various ways known in the art to treat epilepsy and reduce epileptic seizures, and the methods disclosed herein are not limited to only those treatments listed herein.

The present application is further illustrated by the following examples that should not be construed as limiting. The contents of all references, patents, and published patent applications cited throughout this application, as well as the Figures and Tables, are incorporated herein by reference.

### EXAMPLES

Materials & Methods

Patient Inclusion Criteria. Patients with AD who are part of the longitudinal Biomarker patient cohort will be identified by the ADRC clinical core and blood samples obtained. These patients undergo routine csf evaluation. This part of the study will be performed in collaboration with the ADRC staff and MSM memory clinic. Patients will be identified, and de-identified, by clinical data obtained. Medical records will be reviewed to determine biomarker and PET imaging status. Controls will consist of patients in the database with a negative csf biomarker profile (either with or without dementia or MCI), also identified by The ADRC clinical core. Patients must be older than 21 years old and give informed consent (via nurse coordinator). Race will be accepted as self-described (a blood sample may be used for ancestry DNA analysis). Patient data, and imaging data (CT/MRI/PET results) and biomarker status (Ab, Tau and p-Tau) to be extracted from medical records and stored in a database for additional correlative analysis, including age, weight, height (BMI), sex, race, blood pressure, heart rate, body temperature, medications, routine admission blood studies, and drug screens.

Patient Exclusion Criteria. The following will result in exclusion: history of cancer, except basal cell carcinoma; history of stem cell transplant; hemorrhagic avm; brain aneurysm or sub arachnoid hemorrhage; malignant hypertension with acute cardiac, renal, or other non-CNS end organ signs/symptoms; evidence of septic cerebral embolus; Acute myocardial infarction; Active or recent (within 30 days) bleeding diathesis; Anti-coagulation with INR>3; Evidence of sepsis or DIC; Blood glucose<30 or >400 mg/dL, creatinine>4 mg/dL, Hct<25%, Chronic dialysis; Current neurological or psychiatric disease confounding neurological evaluations; Participation in any investigational drug treatment within previous 90 days, or gene or cellular therapy at any time. These inclusion/exclusion criteria match studies of human blood genomics (Jickling G C, et al. RNA in blood is altered prior to hemorrhagic transformation in ischemic stroke. Ann Neural. 2013; 74(2):232-40; Jiclding G C, et al. Signatures of cardioembolic and large-vessel ischemic stroke. Ann Neural. 2010; 68(5):681-92).

Data Management and Storage. All patient associated data will be stored in an electronic database format using the RED-Cap platform. This platform will enable the researchers to maintain a de-identified database of patient information, as well as enabling alert messages to be sent to the coordinator if an event occurs.

RNA-Seq Library Preparation. RNA-Seq libraries are prepared as in preliminary studies. Blood (3.0 mL) is drawn into PAXgene blood collection (Rainen L, et al. Stabilization of mRNA expression in whole blood samples. Clin Chem. 2002; 48(11):1883-90), and processed in batches of eight (Pre-AnalytiX™). RNA quality is verified using a Bioanalyzer (RNA chip, Agilent Bioanalyzer 2100) prior to library preparation. Only samples with an A260/A280 ratio>2.0 and a 28S/18S RNA ratio>5 are subjected to further analysis. The expected total RNA yield from whole blood is 3-8 μg/3 mL of whole blood. RNA (2 μg) is subjected to ribosomal RNA depletion. ERCC spike in libraries will assess library preparation. RNA-Seq Libraries are prepared for the Ion Torrent sequencer using the Ion Total RNA-seq Kitv2. The libraries are verified with a Bioanalyzer chip (DNA Nano Kit, Agilent). Templates are prepared using the Ion One Touch system. Samples are run in batches of four, across two-three chips to give an estimated read depth of 40 million reads/sample. Samples undergo 200 bp sequencing using Ion 540 chips (540). Following sequencing, reactions are analyzed to ensure appropriate base and GC content distribution. The goal is to obtain 20-40M aligned reads/blood sample in this study, based on depth analysis.

Data Alignment: Sequencing data is aligned to the human reference genome using STAR and Bowtie2 (part of the Ion Torrent software). All subsequent analysis is performed using the Tuxedo suite and Partek Genomics Suite v 7.0 running on a dedicated Dell Precision T7600 workstation. RNA-Seq data files (BAM files) are used to generate gene expression values (reads). Exon expression and transcript expression values are also calculated. Once gene read values are determined, they will be transferred to a database for storage. Sequencing data are stored on a local sever prior to upload to NIH. A de-identified database of clinical phenotype data is maintained alongside each transcriptome.

The following quality controls are applied for the data: Only samples with a full diagnosis (and consent) will be considered for sequencing. The average yield is 2-4 ug/3 ml blood sample. The RNA must satisfy the following criteria A260/A280 ratio>2.0 and a 28S/l 8S RNA ratio>5. Following library building, only libraries with >80% of the library in the correct weight size (l 50-400 bp) will be used. (Libraries can be subjected to additional cycles of AmpureXL bead clean up). Following sequencing, samples are ensured to have at least 20 million usable (aligned) reads. If a sample does not create sufficient reads, the library will be re-sequenced. If after two attempts, there is a failure to obtain sufficient quality reads, the sample will be excluded.

Power Analysis: With respect to sample size calculations, statistical analysis in genomic studies poses unique challenges; the threshold for fold change and statistical significance are frequently arbitrary decisions. Power analysis of the pilot RNA-Seq AD data using Partek for small but significant changes in gene expression, (FDR test (a=0.000001, power 1−=0.95), showed a total estimated sample size of 90 will detect nearly 90% of 1.25 fold changes. Other studies show differential gene expression at low levels of fold change are possible with more modest sample sizes (Hart S N, et al. Calculating sample size estimates for RNA sequencing data. J Comput Biol. 2013; 20(12):970-8) However, herein the focus is on pattern analysis, rather than attributing significance to individual gene changes.

Biomarker Assessment. Biomarkers are measured at the Emory ADRC Biomarker Core. Qualitative levels (positive or negative) are used, via the predetermined thresholds set by the unit, in particular whether they are above or below the threshold levels.

Diagnosis Modeling. Expression modeling will determine the pattern of gene expression in the acute setting (upon admittance blood sample) that best discriminates patient's csf biomarker results (diagnosis). Data are normalized and expression data are trained using the PAM module of Partek. The Support Vector Machine model (SVM) is utilized, as this was most effective in previous studies (we need a larger cohort to use neural network modeling). Data are partitioned using “full leave one out” method, and the clinical diagnosis (biomarker level) is used as the prediction value. RNAs for modeling will be selected by performing multifactorial analysis of variance, compensating for age and sex. Models with the highest normalized correct rate are further validated using bootstrap and two-level cross validation.

The initial dataset of 60 samples (40 AD patients and 20 controls) will be subjected to testing and MODELING to identify the best predictive models. Combinations are investigated in a sequential manner, to identify the model with the highest normalized correct diagnostic rate (accuracy (area under the curve), specificity and specificity). The best model will be forwarded to validation studies in future analyses.

MODELING to identify the best predictive models. Combinations are investigated in a sequential manner, to identify the model with the highest normalized correct diagnostic rate (accuracy (area under the curve), specificity and specificity). The best model will be forwarded to validation studies in future analyses.

RNAs differentially expressed are determined and then those expression values are used to generate models to predict the single and combined results of the biomarker assessments.

Validation: an additional 30-40 blood samples are analyzed from the ADRC and MSM to determine whether the biomarker approach can identify the biomarker status in an independent validation group. The best performing models from the testing phase (based on accuracy, sensitivity and specificity) are then tested against this mixed sample set. Accuracy is determined by the AUC/overall accuracy, sensitivity and specificity measures (determined using partek software as previously described (Hardy J J, et al. Assessing the accuracy of blood RNA profiles to identify patients with post-concussion syndrome: A pilot study in a military patient population. PLoS One. 2017; 12(9)).

Interpretation. New NIH guidelines suggest the use of AT(N) biomarker status for research (Jack C R, Jr., et al. NIA-AA Research Framework: Toward a biological definition of Alzheimer's disease. Alzheimers Dement. 2018; 14(4):535-62). Blood based RNA-Seq profiles are an acceptable surrogate biomarker for these current standards. The prediction of both individual csf biomarker levels and an overall biomarker diagnosis is evaluated. These models will be cross validated using the other dementia and MCI data sets. To determine their ability to correctly identify A+T+N+ patients from these other groups. Specifically, models with an accuracy of greater than 90%, and ideally 95% are selected.

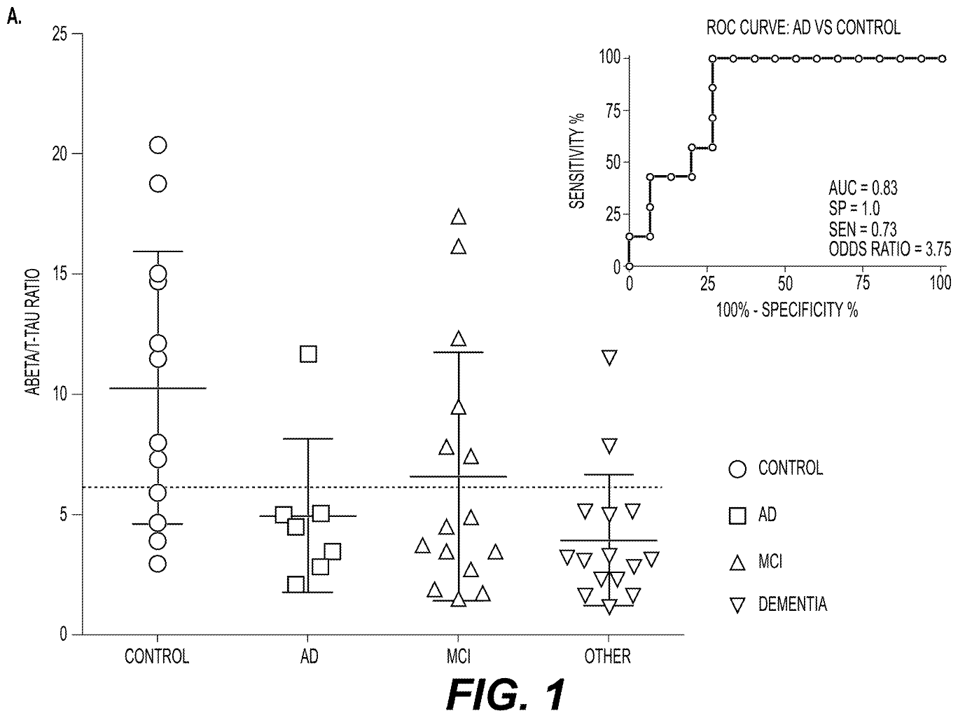

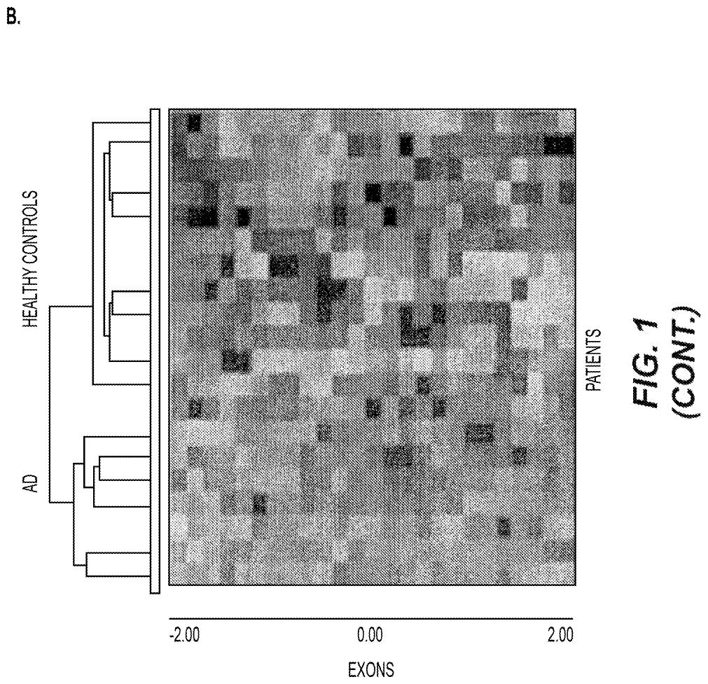

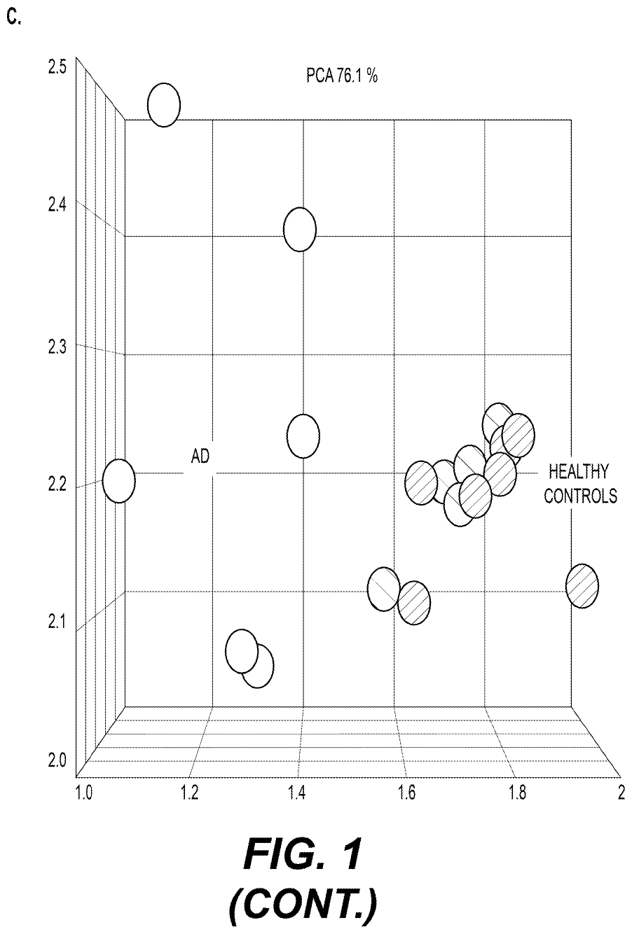

Patients who were clinically diagnosed as having Alzheimer's disorder, other forms of dementia, MCI or healthy controls were recruited and consented to give a blood sample. RNA-Seq libraries were assembled, sequenced on an Ion Torrent S5 sequencer, and aligned to the Hg19 reference genome. Samples had on average 20 million aligned reads, and there were no significant differences in read number between samples, nor mapping (see FIG. 1). Differential expression was determined using Partek on normalized data values, and differentially expressed genes were used for subsequent analysis and modeling. example 1. Blood RNA profiles distinguish between healthy controls and AD patients

Analysis of differentially expressed genes using hierarchical cluster and principle component analysis (PCA) of blood RNA profiles show a clear difference in profiles between controls and AD patients (FIG. 1). PCA reveals the majority of the variation in the group can be attributed to AD status (76.1%). These data were then subjected to K-nearest neighbor modeling, with one level cross validation. This revealed a model with 92.9% accuracy (AUC) in predicting the clinical status of the patient (AD vs healthy control). The data was further subjected to support vector machine modeling, which had a 100% accuracy rate (radial kernel function, 20 variables, gamma 0.01).

### Example 2. Blood RNA Profiles Distinguish Between AD and MCI Patients

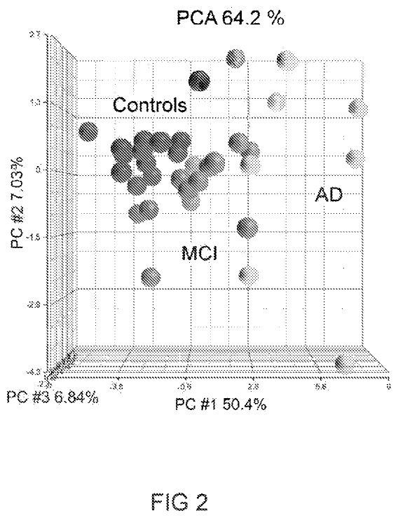

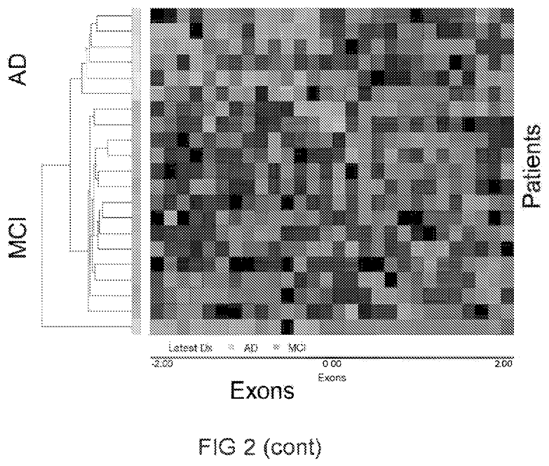

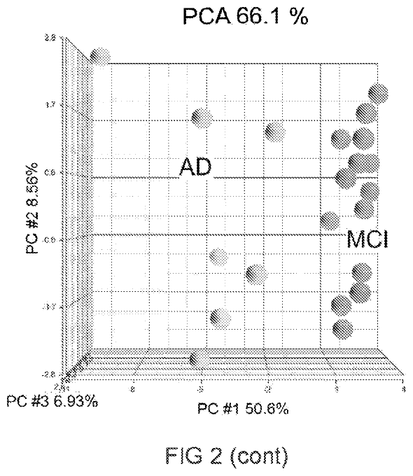

The AD patient selective model was used and MCI data was added to this model for PCA analysis (FIG. 2, Panel C). As can be seen there is a distinct grouping of the MCI patients in-between the healthy controls and the AD patients.

A separate analysis was performed to identify differences between the ad patient group and the MCI group. There was a clear difference in profiles between MCI and AD patients. PCA reveals the majority of the variation in the group can be attributed to AD status (68.1%). These data were then subjected to K-nearest neighbor modeling, with one level cross validation. This revealed a model with 92.9% accuracy (AUC) in predicting the clinical status of the patient (AD vs. MCI).

### Example 3. Blood RNA Profiles Distinguish Between AD Patients and Patients with Other Forms of Dementia

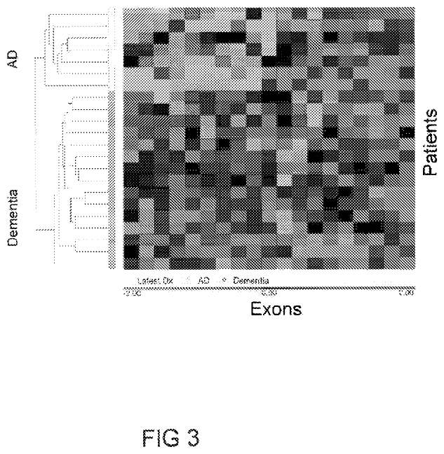

Clinically it is challenging to identify AD from other forms of dementia. The patient cohort consisted of frontal temporal dementia, CADASIL, and other non-specified forms of dementia. Analysis of differentially expressed genes using hierarchical cluster and principle component analysis of blood RNA profiles show a clear difference in profiles between dementia and AD patients (FIG. 3). PCA reveals the majority of the variation in the group can be attributed to AD status (64.5%). These data were then subjected to K-nearest neighbor modeling, with one level cross validation. This revealed a model with 96.7% accuracy (AUC) in predicting the clinical status of the patient (AD vs Dementia). These data show that the RNA profiles generated from high-throughput sequencing of collected blood samples have remarkable accuracy for clinical status prediction.

### Example 4. Alignment of RNA-Seq Data to African American Pan-Genome and Generation of Blood Specific Gtf Files

RNA-seq data from was aligned to the pan-African American genome using Bowtie2. Alignment metrics show approximately 0.2% of reads have unique alignment to the pan-genome. This additional reference can be used with the data to enhance detection of novel RNAs in the AD cohort. Reference genomes are based on known and predicted RNA sequences; unpredicted RNAs must be identified via direct sequencing studies and such novel transcripts are highly tissue dependent. Cufflinks was used to create a blood specific annotation guide from the blood samples. This increases the number of detectable and quantifiable RNAs from 196,398 to over 208,000 transcripts for a novel blood transcriptome. These data show the ability to align to the Pan-Genome and generate novel annotation guides.

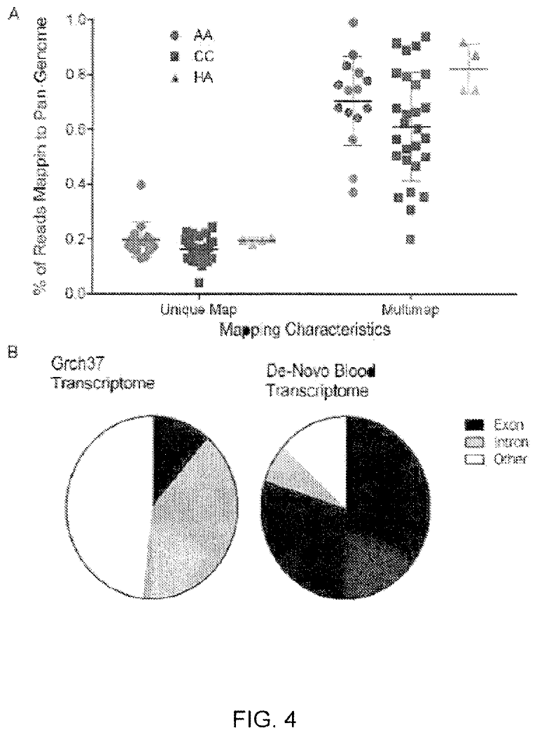

These data show the capability to perform sequencing of collected blood samples, and that RNA profiles have remarkable accuracy for clinical status prediction. The data shows an AUC of 0.93 in modeling tests of exon expression data (FIG. 1) (quantifying coding RNA, miRNA, non-coding RNA, and novel RNAs). In addition, the pan African American genome reference can be utilized in analysis, and data shows novel blood specific RNA annotation guides enhance discrimination of AD from control populations (FIG. 4). This technological approach offers a significant advance from current approaches to identify blood RNA biomarkers for AD diagnosis and treatment.

The present disclosure enables a blood test for AD, which identifies patients earlier who are at very high risk of developing AD. These patients can be identified when their symptoms are less severe, so any therapy may be able to at least halt the progression of the disease. The blood test is effective in these patients and can be a routine screening tool for all people aged 40, and then every ten years thereafter to identify if they have the signature indicative of the AD process.

### Example 5: Characterizing the Temporal Profile of Blood Transcriptome Response Following an EEG Confirmed Seizure

Blood is obtained from patients undergoing video EEG monitoring in an epilepsy-monitoring unit. Blood is collected at baseline and following the occurrence of an EEG confirmed seizure or psychogenic non-epileptic seizures at 6 h, at 24 h and at 72 h post-event. Blood is subjected to RNA sequencing to identify temporal profiles of RNA expression following the seizure. Gene expression profiles are observed to change following the onset of the seizure with some genes increasing or decreasing transiently, and others changing for the entire duration. Example 6: Statistical and bioinformatics analysis to identify the most accurate set of RNA expression patterns to determine seizure occurrence, temporal profile and persistence of the transcriptome response

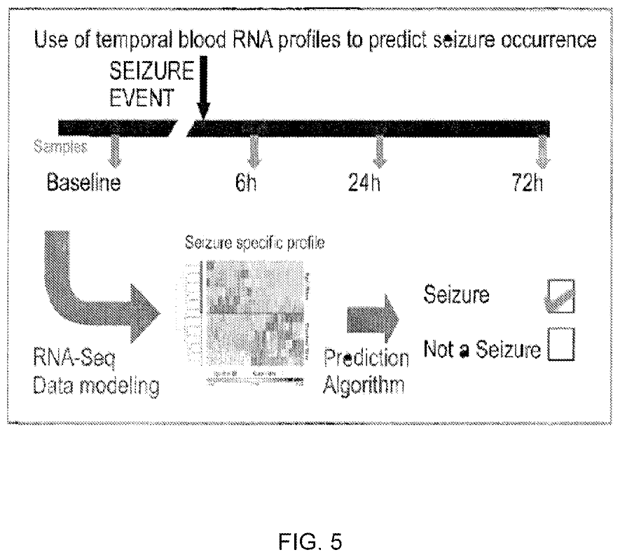

Temporal profiles of whole blood RNA signatures are different in patients following an epileptic seizure and those with psychogenic non-epileptic seizures (FIG. 5).

RNA expression patterns are compared between patients with epileptic seizures and those with psychogenic non-epileptic seizures. Using mathematical models, signatures of RNA are identified that are the most effective classifiers to discriminate between patients with EEG confirmed seizures and non-epileptic seizures. Data are analyzed to determine temporal profiles that predict time of seizure. A resulting diagnostic panel identifies with over 90% accuracy that a seizure event occurred. This data distinguishes epileptic seizure from non-seizure events. Diagnosed epilepsy patients (or patients who suffered a seizure), receive improved medical treatment by reducing diagnosis delay, cost, and unnecessary medication.

The contents of all references, patents, and published patent applications cited throughout this application, as well as the Figures and Tables, are incorporated herein by reference.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims.

What is claimed is:

1. A method of pre-clinical detection for incipient neurodegenerative disease, comprising the steps of:
extracting a whole blood sample from a subject;
preparing an RNA library from the whole blood sample;
sequencing the RNA library;
determining differential expression of a plurality of RNA sequences comprised within the RNA library, wherein the plurality of RNA sequences comprises non-coding RNA (ncRNA);
creating a blood RNA transcriptome profile based on the differential expression of the RNA sequences;
comparing the blood RNA transcriptome profile to a reference blood RNA transcriptome profile derived from a subject with neurodegenerative disease;
detecting incipient neurodegenerative disease based on the correspondence between the blood RNA transcriptome profile and the reference profile derived from a subject with neurodegenerative disease,
wherein the neurodegenerative disease is one or more selected from the group consisting of Huntington's disease, Parkinson's disease, trinucleotide repeat disorders (DRPLA, SBMA, SCA1, SCA2, SCA3, SCA6, SCA7, SCA17, FRAXA, FXTAS, FRAXE, FRDA, DM1, SCA8, SCA12), amyotrophic lateral sclerosis and Batten disease.

2. A method of enhancing treatment of preclinical Parkinson's disease, comprising the steps of:
extracting a whole blood sample from a subject;
preparing an RNA library from the whole blood sample;
sequencing the RNA library;
determining differential expression of a plurality of RNA sequences comprised within the RNA library, wherein the plurality of RNA sequences comprises non-coding RNA (ncRNA);
creating a blood RNA transcriptome profile based on the differential expression of the RNA sequences;
comparing the blood RNA transcriptome profile to a reference blood RNA transcriptome profile derived from a subject with preclinical Parkinson's disease;
detecting preclinical Parkinson's disease based on the correspondence between the blood RNA transcriptome profile and the reference profile derived from a subject with preclinical Parkinson's disease;
treating the subject with a therapy for Parkinson's disease.

3. The method of claim 2, where the therapy for Alzheimer's disease comprises:
administering one or more drugs selected from the group consisting of levodopa, carbidopa, dopamine agonists, catechol O-methyltransferase (COMT) inhibitors, anticholinergics, amantadine, aducanumab and monoamine oxidase type B (MAO-B) inhibitors.
